(19) United States
(12) Patent Application Publication
(10) Pub. No.: US 2008/0114243 A1
(43) Pub. Date: May 15, 2008

(54) ULTRASONIC DIAGNOSTIC APPARATUS, ULTRASONIC DIAGNOSTIC METHOD, AND IMAGE PROCESSING PROGRAM FOR ULTRASONIC DIAGNOSTIC APPARATUS

(75) Inventor: Masato OONUKI, Otawara-Shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS, Otawara-Shi (JP)

(21) Appl. No.: 11/937,750

(22) Filed: Nov. 9, 2007

(30) Foreign Application Priority Data
Nov. 10, 2006 (JP) 2006-304816

(51) Int. Cl. A61B 8/00

(52) U.S. Cl. 600/443; 382/131

(57) ABSTRACT

The present invention relates to an ultrasonic diagnostic apparatus, an ultrasonic diagnostic method, and an image processing program for the ultrasonic diagnostic apparatus. An image reconstruction unit converts B-mode image data and doppler mode image data into volume data with the common coordinate axes. A calculation unit calculates the estimated volume of a fetus based upon the volume data, and calculates the estimated weight of the fetus based upon a coefficient stored beforehand in a data storage unit and the estimated volume of the fetus thus calculated. A display unit displays the calculation results with respect to the estimated weight of the fetus etc. The ultrasonic diagnostic apparatus, the ultrasonic diagnostic method, and the image processing program for the ultrasonic diagnostic apparatus according to the present invention improves the operability of the ultrasonic diagnostic apparatus for calculating the estimated weight of a fetus.

### BACKGROUND OF THE INVENTION

1. Field of the Invention

The present invention relates to an ultrasonic diagnostic apparatus, an ultrasonic diagnostic method, and an image processing program for the ultrasonic diagnostic apparatus. In particular, the invention relates to an ultrasonic diagnostic apparatus, an ultrasonic diagnostic method, and an image processing program for the ultrasonic diagnostic apparatus allowing the improvement of an operability.

2. Related Art

Ultrasonic diagnostic apparatuses employ ultrasonic waves, which provides the advantage of involving no risk due to exposure to radiation. Accordingly, in recent years, ultrasonic diagnostic apparatuses have come to be employed in diagnostic procedures performed on the fetus, as the fetus is easily affected by exposure to radiation.

Conventionally, in addition to diagnostic procedures performed on a fetus using such an ultrasonic diagnostic apparatus, a method for calculating the estimated weight of the fetus is known using the ultrasonic diagnostic apparatus, which allows surgeons, medical technicians, etc., (who will be referred to as “operators” hereafter) to observe the growth of the fetus.

With conventional methods for calculating the estimated weight of a fetus, the length of the head, the length of the abdomen, and the length of the legs of the fetus in the mother's body are measured, and the current weight of the fetus is calculated.

Specifically, first, the operator instructs the ultrasonic diagnostic apparatus to display tomographic images in increments of parts of the body of the fetus. As shown in FIG. 1, the operator instructs the ultrasonic diagnostic apparatus to display a tomographic image of the head of the fetus, and measures the BPD. As shown in FIG. 2, the operator instructs the ultrasonic diagnostic apparatus to display a tomographic image of the head of the fetus, and measures the HC. As shown in FIG. 3, the operator instructs the ultrasonic diagnostic apparatus to display a tomographic image of the abdomen of the fetus, and measures the AC. As shown in FIG. 4, the operator instructs the ultrasonic diagnostic apparatus to display a tomographic image of the femoral region of the fetus, and measures the FL.

Next, the measurement results obtained as shown in FIGS. 1 to 4 is substituted into a predetermined estimated fetal weight (EFW) calculation equation. As a result, the estimated weight of the fetus is calculated, thereby the calculation results is displayed as shown in FIG. 5.

However, conventional estimated weight calculation methods have a matter in that the operator must perform measurements based upon the tomographic images as necessary to calculate the estimated weight of the fetus while displaying these tomographic images in sequence. Furthermore, high-precision calculation of the estimated weight requires suitably precise tomographic images. In order to select such suitable tomographic images, the operator needs to repeatedly operate the operation panel, which is troublesome.

In order to avoid this troublesome operation, a method is conceivable in which the tomographic image of each part of the fetus is selected every time the operator performs a single display operation, and the estimated weight of the fetus is calculated based upon the tomographic images thus selected. However, this method has the following problem. In a case in which the tomographic image of the head of the fetus is displayed, and the BPD is measured based upon the tomographic image thus displayed, in some cases, the tomographic image thus displayed according to such a single display operation is not a suitable tomographic image that is perpendicular to the axis of the head of the fetus (a so-called tomographic image in an axial plane), but is a tomographic image obtained by scanning the head of the fetus at a somewhat oblique angle with respect to the tomographic image in the axial plane. The elliptical image shown in FIG. 1 is thereby distorted, leading to two-dimensional deviation in the measurement. As a result, the estimated weight of the fetus cannot be calculated with high precision.

Furthermore, the operator needs to repeatedly perform the operation for each of a multiple number of items before calculating the estimated weight of the fetus. With such an arrangement, in some cases, the operator may neglect to perform a necessary operation from among these necessary items. In such a case, even if the operator performs sufficient operations for most items but neglects to perform a necessary operation, the estimated weight of the fetus cannot be calculated, and the operator must perform the calculation procedure again for calculating the estimated weight of the fetus, which is troublesome.

### SUMMARY OF THE INVENTION

The present invention has been made in view of such a situation. Accordingly, it is an object of the present invention to provides an ultrasonic diagnostic apparatus, an ultrasonic diagnostic method, and an image processing program for the ultrasonic diagnostic apparatus allowing the improvement of an operability in case of calculating the estimated weight of a fetus.

In order to solve the aforementioned problems, an ultrasonic diagnostic apparatus according to an aspect of the present invention comprises: a volume data generation unit configured to oscillate a plurality of ultrasonic wave transducer elements to transmit ultrasonic waves and to receive reflection waves which are reflected from a subject body and generate volume data on the basis of reception signals obtained by converting the reflection waves by the ultrasonic wave transducer elements; a three-dimensional image data generation unit configured to generate three-dimensional image data on the basis of the volume data; and an estimated weight calculation unit configured to calculate the estimated weight of the subject body on the basis of the volume data.

In order to solve the aforementioned problems, an ultrasonic diagnostic method according to another aspect of the present invention comprises: a volume data generation step for oscillating a plurality of ultrasonic wave transducer elements to transmit ultrasonic waves and receiving reflection waves which are reflected from a subject body and generating volume data on the basis of reception signals obtained by converting the reflection waves by the ultrasonic wave transducer elements; a three-dimensional image data generation step for generating three-dimensional image data on the basis of the volume data; and an estimated weight calculation step for calculating the estimated weight of the subject body on the basis of the volume data.

In order to solve the aforementioned problems, an image processing program for an ultrasonic diagnostic apparatus according to yet another aspect of the present invention instructs a computer to execute: a volume data generation step for oscillating a plurality of ultrasonic wave transducer elements to transmit ultrasonic waves and receiving reflection waves which are reflected from a subject body and generating volume data on the basis of reception signals obtained by converting the reflection waves by the ultrasonic wave transducer elements; a three-dimensional image data generation step for generating three-dimensional image data on the basis of the volume data; and an estimated weight calculation step for calculating the estimated weight of the subject body on the basis of the volume data.

### BRIEF DESCRIPTION OF THE DRAWINGS

FIG. 1 is an explanatory diagram for describing a conventional calculating method for the estimated weight of a fetus;

FIG. 2 is an explanatory diagram for describing a conventional calculating method for the estimated weight of a fetus;

FIG. 3 is an explanatory diagram for describing a conventional calculating method for the estimated weight of a fetus;

FIG. 4 is an explanatory diagram for describing a conventional calculating method for the estimated weight of a fetus;

FIG. 5 is an explanatory diagram for describing a conventional calculating method for the estimated weight of a fetus;

FIG. 6 is a block diagram which shows an internal configuration of an ultrasonic diagnostic apparatus according to the present invention;

FIG. 7 is a flowchart for describing an estimated weight calculation processing performed by the ultrasonic diagnostic apparatus shown in FIG. 6;

FIG. 8 is an explanatory diagram for describing the state of a subject body fetus in the amniotic fluid stored in the womb; and

FIG. 9 is a diagram which shows a display example in which the estimated weight of a fetus is displayed on a display unit shown in FIG. 6.

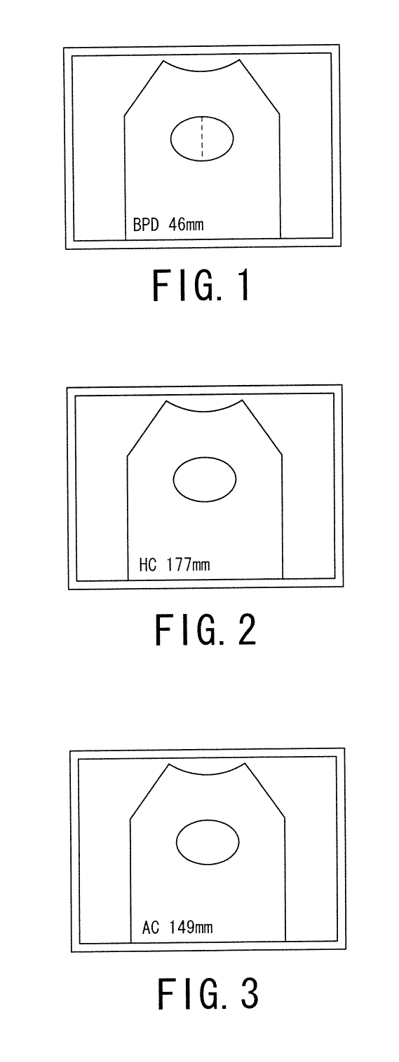

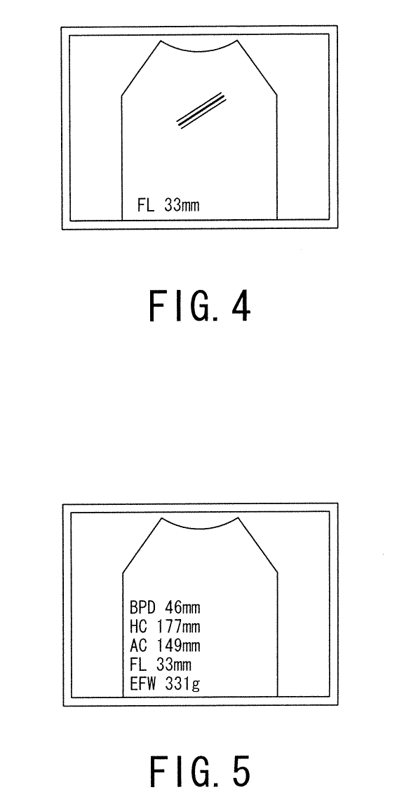

### DESCRIPTION OF THE PREFERRED EMBODIMENTS

Description will be made regarding an embodiment of the present invention with reference to the drawings.

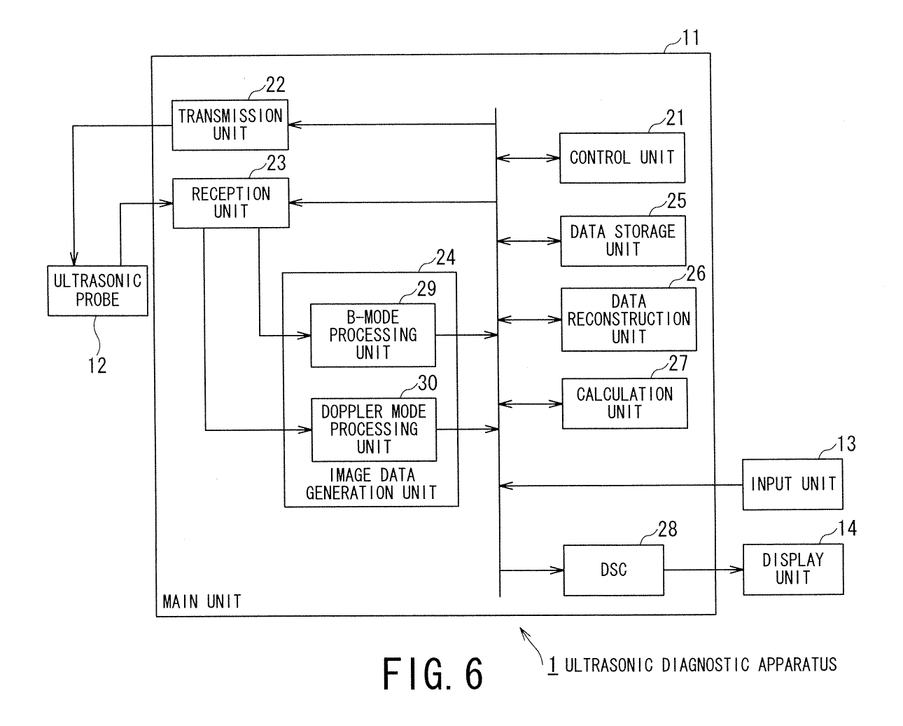

FIG. 6 shows an internal configuration of an ultrasonic diagnostic apparatus 1 according to the present invention.

The ultrasonic diagnostic apparatus 1 comprises a main unit 11, an ultrasonic probe 12 which is connected to the main unit 11 via an electronic cable, an input unit 13, and a display unit 14.

As shown in FIG. 6, the main unit 11 of the ultrasonic diagnostic apparatus 1 comprises a control unit 21, a transmission unit 22, a reception unit 23, an image data generation unit 24, a data storage unit 25, an image reconstruction unit 26, a calculation unit 27, and a DSC (Digital Scan Converter) 28. It should be noted that the control unit 21, the transmission unit 22, the reception unit 23, the image data generation unit 24, the data storage unit 25, the image reconstruction unit 26, the calculation unit 27, and the DSC 28 are mutually connected via a bus in the main unit 11 of the ultrasonic diagnostic apparatus.

The control unit 21 comprises a CPU (Central Processing Unit) or an MPU (Micro Processing Unit), ROM (Read Only Memory), RAM (Random Access Memory), and the like. The control unit 21 generates various control signals, and supplies the control signals thus generated to the respective units, thereby centrally controlling the operation of the ultrasonic diagnostic apparatus 1.

The transmission unit 22 comprises a rate pulse generator, a transmission delay circuit, and a pulse generator (none of which are shown). The rate pulse generator generates a rate pulse, which determines the pulse repetition frequency of the ultrasonic pulses to be input to the internal body region of the subject body, based on a control signal supplied from the control unit 21, and outputs the rate pulse thus generated to the transition delay circuit. The transition delay circuit is a delay circuit which provides a function of setting the focal point and the deflection angle of the ultrasonic beam to be transmitted. Specifically, the transmission delay circuit sets a delay time for the rate pulse supplied from the rate pulse generator, based on the control signal supplied from the control unit 21, so as to set to desired values the focal point and the deflection angle of the ultrasonic beam that is to be transmitted. The transmission delay circuit supplies the rate pulse thus set to the pulse generator. The pulse generator is a driving circuit which generates high voltage pulses for driving ultrasonic oscillators. The pulse generator generates high voltage pulses for driving the ultrasonic oscillators according to the rate pulse supplied from the transmission delay circuit, and outputs the high voltage pulses thus generated to the ultrasonic probe 12.

The reception unit 23 comprises a pre-amplifier, a reception delay circuit, and an adder (none of which are shown). The pre-amplifier acquires a reception signal that is based on the reflected waves of the ultrasonic pulses applied to the subject body via the ultrasonic probe 12. The pre-amplifier amplifies the reception signal thus acquired up to a predetermined level, and supplies the reception signal thus amplified to the reception delay circuit.

The reception delay circuit sets a delay time for the reception signal, which has been amplified by and supplied from the pre-amplifier, based on a control signal supplied from the control unit 21. Here, the delay time is set, in increments of ultrasonic oscillators, to a value that corresponds to the difference in the propagation time of the ultrasonic wave from the focal point. The reception delay circuit supplies the reception signal thus set to the adder. The adder adds together the reception signals which have been supplied from the reception delay circuit in increments of ultrasonic oscillators, and supplies the added reception signal to the image data generation unit 24.

The image data generation unit 24 comprises a B-mode processing unit 29 and a Doppler mode processing unit 30. The B-mode processing unit 31 comprises a logarithmic amplifier, an envelope detection circuit, and a TGC (Time Gain Control) circuit (none of which are shown). The image data generation unit 24 performs the following processing according to a control signal supplied from the control unit 21.

That is to say, the logarithmic amplifier of the B-mode processing unit 31 logarithmically amplifies the reception signal supplied from the reception unit 23, and supplies the reception signal thus logarithmically amplified to the envelope detection circuit. The envelope detection circuit is a circuit which detects only the amplitude without detecting the ultrasonic frequency component. Specifically, the envelope detection circuit detects the envelope of the reception signal supplied from the logarithmic amplifier, and supplies the reception signal thus detected to the TGC circuit. The TGC circuit adjusts the magnitude of the reception signal supplied from the envelope detection circuit so as to generate an image with sufficiently uniform brightness in the final stage, thereby generating B-mode image data. The B-mode image data thus generated is supplied to the data storage unit 25.

The Doppler mode processing unit 30 comprises a reference signal generator, a π/2 phase sifter, a mixer, an LPF (Low Pass Filter), a Doppler signal storage circuit, an FFT (Fast Fourier Transform) analyzer, a computing unit, etc. (none of which are shown). The Doppler mode processing unit 30 mainly performs quadratic phase detection and FFT analysis. The Doppler mode image data thus generated is supplied to the data storage unit 25.

The data storage unit 25 comprises an HDD (Hard Disc Drive) or the like. The data storage unit 25 acquires the B-mode image data supplied from the B-mode processing unit 31 and the Doppler mode image data supplied from the Doppler mode processing unit 32, and stores the B-mode image data and the Doppler mode image data thus acquired. The data storage unit 25 supplies the B-mode image data and the Doppler mode image data thus stored to the image reconstruction unit 26 and the DSC 28 as necessary according to an instruction from the control unit 21.

Furthermore, the data storage unit 25 acquires volume data and various kinds of three-dimensional image data supplied from the image reconstruction unit 26, and stores the volume data and the various kinds of three-dimensional data thus acquired. Moreover, the data storage unit 25 supplies the volume data and the various kinds of three-dimensional image data thus stored to the calculation unit 27 and the DSC 28 as necessary. Furthermore, the data storage unit 25 stores the calculation results supplied from the calculation unit 27, and supplies the calculation results thus stored to the DSC 28 as necessary. In addition, the data storage unit 25 stores predetermined coefficient (which is a value with respect to the density and which is used for calculating the estimated weight of the fetus based upon the estimated volume of the fetus), and supplies the predetermined coefficient thus stored to the calculation unit 27 as necessary.

The image reconstruction unit 26 reads out the B-mode image data and the Doppler mode image data thus stored in the data storage unit 25 under the control of the control unit 21, and transforms the B-mode image data and the Doppler mode image data thus read out into volume data having common coordinate axes, and supplies the volume data thus transformed to the data storage unit 25. The image reconstruction unit 26 performs reconstruction processing using various kinds of computation processing based upon the volume data thus transformed, thereby generating various kinds of three-dimensional image data. The various kinds of three-dimensional image data thus generated are supplied to the data storage unit 25.

The calculation unit 27 reads out the volume data stored in the data storage unit 25 under the control of the control unit 21, and calculates the estimated volume of the fetus based upon the volume data thus read out. Under the control of the control unit 21, the calculation unit 27 reads out predetermined coefficient (which is a value with respect to the density and which is used for calculating the estimated weight of the fetus based upon the estimated volume of the fetus) stored beforehand in the data storage unit 25. Then, the calculation unit 27 calculates the estimated weight of the fetus based upon the predetermined coefficient thus read out and the estimated volume of the fetus thus calculated, and supplies the calculation result to the data storage unit 25.

The DSC 28 acquires the data sets comprising the B-mode image data and the Doppler mode image data or the three-dimensional image data supplied from the data storage unit 25 under the control of the control unit 21. Then, the DSC 28 converts the data format of the B-mode image data and the Doppler mode image data or the three-dimensional image data from the ultrasonic scanning line format to the video scanning line format. Furthermore, the DSC 28 performs predetermined image processing or computation processing for the image data thus converted, and supplies the image data thus processed to the display unit 14. Moreover, the DSC 28 acquires the calculation result with respect to the estimated weight of the fetus supplied from the data storage unit 25. Then, the DSC 28 converts the data format of the calculation result with respect to the estimated weight of the fetus thus acquired to the video scanning line format, and performs the predetermined image processing or computation processing for the calculation result thus converted. The DSC 28 supplies the calculation result thus processed to the display unit 14.

The ultrasonic probe 12 is an ultrasonic transducer which is connected to the main unit 11 via an electronic cable, and which transmits/receives ultrasonic waves when the front face of the ultrasonic probe 12 is in contact with the surface of the subject body. The ultrasonic probe 12 includes a one-dimensional or two-dimensional matrix array of microscopic ultrasonic oscillators (not shown) arrayed on the front end thereof. Each ultrasonic oscillator is an electro-acoustic converter provided in the form of a piezo-electric oscillator. In the transmission step, the ultrasonic probe 12 converts the electric pulses input from the transmission unit 22 of the main unit 11 into ultrasonic pulses (transmission ultrasonic waves). On the other hand, in the reception step, the ultrasonic probe 12 converts the reflected waves reflected from the subject body into an electric signal, and outputs the electric signal thus converted to the main unit 11.

The input unit 13 is connected to the main unit 11 via an electrical cable. The input unit 13 has an operation panel including various input devices. Examples of the input devices include: an estimated weight calculation button which allows the operator to issue an instruction to calculate the estimated weight; and a display panel, a keyboard, a trackball, a mouse, etc., which allow the operator to input various instructions. Such an arrangement allows the operator to input the patient information, the measurement parameters, the physical parameters, the template size, the time phase and the grid spacing of the image which are to be used for the image computation.

The display unit 14 is connected to the DSC 28 of the main unit 11 via a cable. The display unit 14 includes an unshown LCD (Liquid Crystal Display) or an unshown CRT (Cathode Ray Tube). The display unit 14 acquires from the DSC 28 the B-mode image data, the Doppler mode image data, the three-dimensional image data, the calculation result with respect to the estimated weight of the fetus, etc., as converted from the ultrasonic scanning line data format into the video scanning line format. Then, the display unit 14 displays, on the unshown LCD or the unshown CRT, the B-mode image data, the Doppler mode image data, the three-dimensional image data, the calculation result with respect to the estimated weight of the fetus, etc.

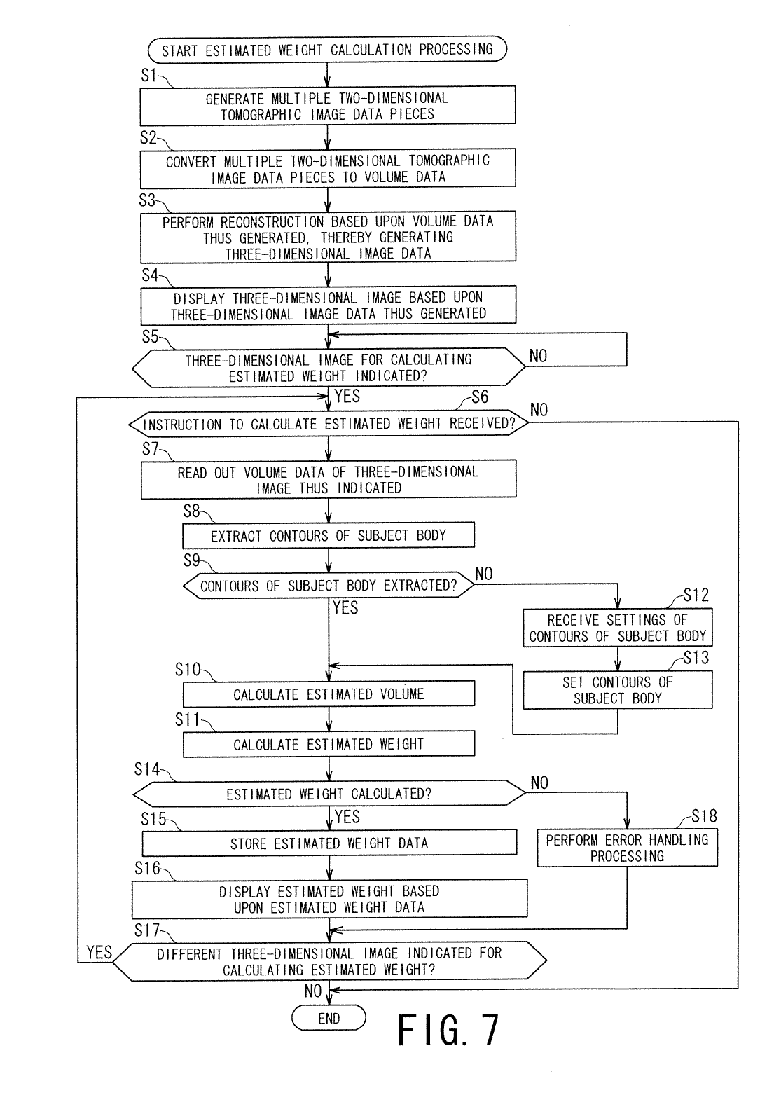

Next, description will be made regarding the estimated weight calculation processing performed by the ultrasonic diagnostic apparatus 1 shown in FIG. 6 with reference to the flowchart shown in FIG. 7. It should be noted that description will be made with reference to the flowchart shown in FIG. 7 regarding the estimated weight calculation processing with reference to a specific example of calculating the estimated weight of a fetus in the amniotic fluid in the womb as the subject body. It is needless to say that the present invention can be applied to various subject bodys other than a fetus in the amniotic fluid in the womb.

In Step S1, the B-mode processing unit 29 and the Doppler mode processing unit 30 of the image data generation unit 24 generate multiple two-dimensional tomographic image data pieces. Specifically, such multiple two-dimensional image data pieces are generated as follows.

The transmission unit 22 transmits an ultrasonic beam to the subject body according to an ultrasonic wave transmission control signal supplied from the control unit 21. That is to say, the rate pulse generator of the transmission unit 22 generates a rate pulse signal determined based upon the ultrasonic wave transmission control signal supplied from the control circuit 21 such that the pulse repetition frequency of the ultrasonic pulses to be input to the internal body region of the subject body is set to a predetermined value. The rate pulse generator supplies the rate pulse signal thus generated to the transmission delay circuit. Then, the transmission delay circuit sets a delay time for the rate pulse signal supplied from the rate pulse generator based upon the ultrasonic wave transmission control signal supplied from the control unit 21 such that the focal point and the deflection angle (θ1) of the ultrasonic beam to be transmitted are set to respective predetermined values. The transmission delay circuit supplies the rate pulse signal thus set to the pulse generator. Then, the pulse generator generates high-voltage pulses based upon the rate pulse signal supplied from the transmission delay circuit for driving the ultrasonic oscillators. The pulse generator outputs the high-voltage pulses thus generated to the ultrasonic probe 12. The ultrasonic probe 12 converts the high-voltage pulses (electric pulses) thus input from the transmission unit 22 into ultrasonic pulses, and transmits the ultrasonic pulses thus converted to the subject body. Some of the ultrasonic waves transmitted into the internal body region of the subject body are reflected from the tissue in the internal body region of the subject body or the interface between the internal organs which have different acoustic impedances.

The ultrasonic probe 12 converts the reflected wave reflected from the subject body into an electric signal, and outputs the electric signal thus converted to the main unit 11. The reception unit 23 amplifies the reception signal input from the ultrasonic probe 12 according to an ultrasonic wave reception control signal supplied from the control unit 21. Furthermore, the reception unit 23 sets a predetermined delay time for the reception signal thus amplified, and supplies the reception signal thus set to the image data generation unit 24. That is to say, the pre-amplifier of the reception unit 23 acquires the reception signal that is based on the reflected wave of the ultrasonic wave that was emitted to the subject body via the ultrasonic probe 12, and amplifies the reception signal thus acquired to a predetermined level. The pre-amplifier supplies the reception signal thus amplified to the reception delay circuit.

The reception delay circuit of the reception unit 23 sets a delay time for the reception signal thus amplified by and supplied from the pre-amplifier according to an ultrasonic wave reception control signal supplied from the control unit 21. Here, the reception delay circuit sets the delay time, in increments of ultrasonic oscillators, to a value that corresponds to the difference in the propagation time of the ultrasonic wave from the focal point. The reception delay circuit supplies the reception signal thus set to the adder. The adder adds the reception signals which have been generated based on each of the ultrasonic oscillators and which have been supplied from the reception delay circuit. The adder supplies the reception signal thus added to the image data generation unit 24.

The B-mode processing unit 31 and the Doppler mode processing unit 32 of the image data generation unit 24 perform various processing for the reception signal supplied from the reception unit 23 so as to generate the B-mode image data and the Doppler mode image data with a deflection angle θ1. The B-mode image data and the Doppler mode image data thus generated with a deflection angle θ1 are supplied to the data storage unit 25.

The data storage unit 25 acquires the B-mode image data and the Doppler mode image data thus generated with a deflection angle θ1, which have been supplied from the B-mode processing unit 31 and the Doppler mode processing unit 32 of the image data generation unit 24, and stores the B-mode image data and the Doppler mode image data with a deflection angle θ1 thus acquired.

Subsequently, the transmission/reception of the ultrasonic waves is performed in the same way according to the above-described procedure every time the transmission/reception direction of the ultrasonic waves is incremented by Δθ in the N direction, thereby providing real-time scanning of the internal body region of the subject body. The real-time scanning is performed over the deflection angle range between θ1 and θ1+(N−1)Δθ. In this step, the control unit 21 issues a control signal which sequentially changes the delay time to be set by the transmission delay circuit of the transmission unit 22 and the delay time to be set by the reception delay circuit of the reception unit 23 to predetermined values that correspond to the current ultrasonic wave transmission/reception direction, thereby generating the B-mode image data pieces and the Doppler mode image data pieces over the deflection angle range between θ1+Δθ and θ1+(N−1)Δθ.

Furthermore, the data storage unit 25 stores the B-mode image data pieces and the Doppler mode image data pieces thus generated over the deflection angle range between θ1+Δθ and θ1+(N−1)Δθ, in addition to the B-mode image data and the Doppler mode image data which have been generated with the deflection angle θ1 and which have already been stored.

As described above, such an arrangement allows the operator to generate a set of a single two-dimensional B-mode image data piece and a single two-dimensional Doppler mode image data piece with a predetermined time phase. Furthermore, such an arrangement allows the operator to store the two-dimensional B-mode data piece and the two-dimensional Doppler mode image data piece thus generated.

Subsequently, the operator performs the above-described operation in the same way under different spatial conditions, thereby acquiring three-dimensional tomographic image data which is composed of multiple two-dimensional tomographic image data pieces (two-dimensional B-mode image data pieces and Doppler mode image data pieces).

Specifically, let us consider a case in which the operator performs manual scanning using the ultrasonic probe 12 having multiple ultrasonic oscillators one-dimensionally arrayed. For example, the operator performs manual scanning by turning the probe 12 around the axis along which the ultrasonic oscillators are one-dimensionally arrayed or by moving the probe 12 in one direction on the surface of the subject body at a constant speed, thereby acquiring three-dimensional tomographic image data that is composed of multiple two-dimensional tomographic image data pieces. It is needless to say that an arrangement may be made in which scanning is performed by mechanically moving the ultrasonic probe 12 having multiple ultrasonic oscillators one-dimensionally arrayed.

Also, an arrangement may be made in which three-dimensional scanning is directly performed using the ultrasonic probe 12 having multiple ultrasonic oscillators two-dimensionally arrayed, thereby acquiring three-dimensional tomographic image data. The present invention can be applied to various arrangements regardless of the scanning method, as long as such arrangements have a function of acquiring three-dimensional tomographic image data.

The multiple two-dimensional tomographic image data pieces (two-dimensional B-mode image data pieces and Doppler mode image data pieces) thus acquired (generated) are sequentially stored in the data storage unit 25.

In Step S2, in accordance with the control of the control unit 21, the image reconstruction unit 26 reads out the multiple two-dimensional B-mode image data pieces and Doppler mode image data pieces stored in the data storage unit 25, and converts the multiple two-dimensional B-mode image data pieces and Doppler mode image data pieces thus read out into volume data having common coordinate axes. The volume data thus converted is supplied to the data storage unit 25.

In Step S3, the image reconstruction unit 26 performs reconstruction processing using various kinds of computation processing based upon the volume data thus converted, thereby generating various kinds of three-dimensional image data using various kinds of methods. The various kinds of three-dimensional image data thus generated are supplied to the data storage unit 25.

In Step S4, under the control of the control unit 21, the DSC 28 acquires the three-dimensional image data which has been generated using various kinds of methods and which has been supplied from the data storage unit 25, transforms the data format of the three-dimensional image data thus generated and thus acquired from the ultrasonic scanning line format to the video scanning line format, performs predetermined image processing or computation processing for the three-dimensional image data thus transformed, and supplies the three-dimensional image data thus processed to the display unit 14. The display unit 14 acquires the three-dimensional image data from the DSC 28, of which data format has been transformed from the ultrasonic scanning line format to the video scanning line format, and displays the three-dimensional image data, which has been processed in various methods, on the unshown LCD or the unshown CRT. Subsequently, the operator performs the above-described operation in the same way, thereby generating multiple different two-dimensional tomographic image data pieces. Thus, multiple three-dimensional stationary images (frozen images) obtained based upon different volume data pieces are sequentially displayed.

In Step S5, the control unit 21 determines whether or not the three-dimensional image for calculating the estimated weight (VOI (Voxel of Interest) which is a three-dimensional image for calculating the estimated weight) is indicated by the operator operating the input unit 13. In this step, the control unit 21 enters the standby state until determination is made that the three-dimensional image for calculating the estimated weight is indicated by the operator operating the input unit 13.

In a case in which determination is made in Step S5 that the three-dimensional image for calculating the estimated weight is indicated by the operator operating the input unit 13, the flow proceeds to Step S6 where the control unit 21 determines whether or not the operator issues an instruction to calculate the estimated weight by operating the estimated weight calculation button (not shown) provided to the input unit 13.

In a case in which determination is made in Step S6 that the operator issues an instruction to calculate the estimated weight by operating the estimated weight calculation button (not shown) provided to the input unit 13, the flow proceeds to Step S7 where the calculation unit 27 reads out the volume data that corresponds to the three-dimensional image, which has been indicated by the operator and which is stored in the data storage unit 25, in accordance with the control of the control unit 21. The image reconstruction unit 26 transforms multiple two-dimensional B-mode data pieces and Doppler mode image data pieces into volume data (voxel data) with common coordinate axes. Here, the volume data is composed of a set of minute cubic data pieces (so-called voxels).

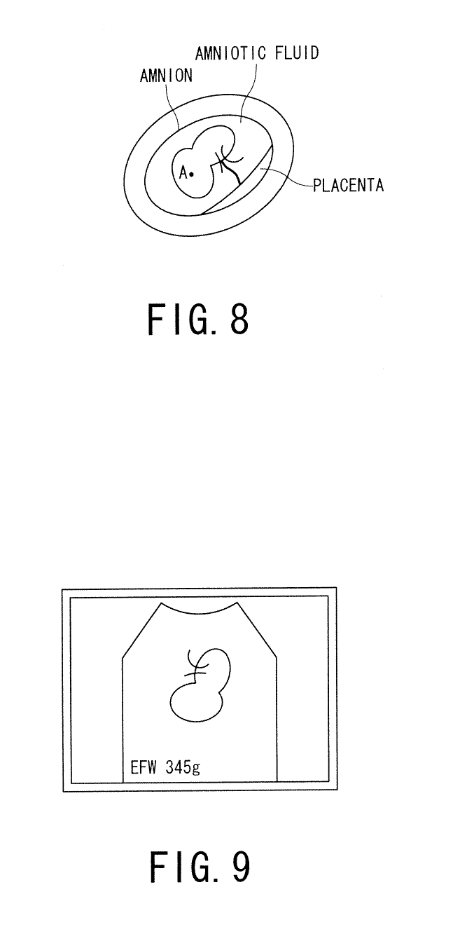

In Step S8, the calculation unit 27 extracts the contours of the subject body (e.g., fetus) based upon the set of minute cubic data pieces (so-called voxels). Specifically, the calculation unit 27 extracts the contours of the subject body (fetus) based upon the brightness value of the volume data (brightness value of each voxel) included in the three-dimensional image for calculating the estimated weight. First, as shown in FIG. 8, before the extraction of the contours of the subject body (fetus) in the amniotic fluid, the operator sets the contour extraction start point to a point around the central portion of the tissue such as the head, the torso, or the like (the contour extraction start point A is indicated in FIG. 8) by operating the input unit 13, for example.

In FIG. 8, the start point is set to the contour extraction start point A. Then, comparison is made between the brightness value of the volume data (the brightness value of the voxel) at a point starting with the contour extraction start point A thus set and the brightness value of an adjacent voxel. This comparison is repeatedly and sequentially performed toward the outer direction in the indicated region of the three-dimensional image for calculating the estimated weight. In this comparison step, determination is made whether or not the change in the brightness value of the volume data (the difference in the brightness value) is lager than a predetermined reference value set beforehand. In a case in which the subject body is a fetus, it can be assumed that the difference in the brightness between the fetus and the amniotic fluid is lager than the predetermined reference value set beforehand. Accordingly, determination is made at the interface between the fetus and the amniotic fluid that the change in the brightness value of the volume data (difference between the brightness values) is lager than the predetermined reference value. On the other hand, in the region of the body of the fetus or in the region of the amniotic fluid, the change in the brightness value of the volume data (difference between the brightness values) is smaller than the predetermined reference value.

In a case in which determination has been made that the change in the brightness value of the volume data (difference between the brightness values) is lager than the predetermined reference value, the voxel that exhibits a higher brightness value is extracted as the voxel that belongs to the body of the subject body (fetus) from among the two voxels that exhibit a difference in the brightness value lager than the predetermined reference value set beforehand. On the other hand, a lower brightness value is extracted as the voxel that belongs to the amniotic fluid. Thus, the interface between the fetus and the amniotic fluid is extracted based upon the voxels thus extracted. That is to say, the interface region is extracted based upon the interface thus extracted. In the interface region thus extracted from the three-dimensional image for calculating the estimated weight, the brightness values of the volume data rapidly change to lower values toward the outer direction. For example, in a case in which the subject body is a fetus, the contour region matches the interface between the fetus and the amniotic fluid. In other words, the contour region thus extracted matches the contours of the subject body fetus.

As described above, the contours of the subject body fetus are extracted based upon the volume data.

In Step S9, the control unit 21 determines whether or not the control unit 21 has extracted the contours of the subject body in the contour extraction processing denoted by Step S8. For example, in some cases, the subject body fetus in the amniotic fluid is in contact with the amnion, and there is little amniotic fluid between the fetus and the amnion. In such a case, it is considered to be difficult to appropriately extract the interface between the fetus and the amnion. In a case in which the interface between the fetus and the amnion cannot be extracted, determination is made that the contours of the subject body have not been extracted in the contour extraction processing denoted by Step S8. On the other hand, in a case in which the interface between the fetus in the amniotic fluid in the normal state and the amnion is appropriately extracted, determination is made that the contours of the subject body have been extracted in the contour extraction processing denoted by Step S8.

In a case in which determination has been made in Step S9 that the contours of the subject body have been extracted in the contour extraction processing, the flow proceeds to Step S10 where the calculation unit 27 calculates the estimated volume of the fetus based upon the volume data read out in accordance with the control of the control unit 21. That is to say, the length of one side of each voxel is known. Accordingly, the voxels included within the contours of the subject body (fetus) thus extracted are integrated, thereby calculating the estimated volume of the fetus.

In Step S11, the calculation unit 27 reads out a predetermined coefficient (which is a value with respect to the density of a fetus, and which is used for calculating the estimated weight of the fetus based upon the estimated volume of the fetus) stored beforehand in the data storage unit 25, and calculates the estimated weight of the fetus based upon the predetermined coefficient thus read out and the estimated volume of the fetus thus calculated. The estimated weight data thus calculated is supplied to the data storage unit 25. It should be noted that such an arrangement allows the operator to set the predetermined coefficient to a desired value, and to change the value thus set. Also, the predetermined parameter may be changed based upon the disease of the fetus (e.g., hydrocephalus). Also, an arrangement may be made in which predetermined coefficients are set beforehand in increments of parts of the subject body (e.g., the head, the torso, etc.), and the estimated weight of the subject body is calculated using the predetermined coefficients thus set.

On the other hand, in a case in which determination has been made in Step S9 that the contours of the subject body have not been extracted in the contour extraction processing, the flow proceeds to Step S12 where the control unit 21 allows the operator to operate the input unit 13 so as to indicate the contours of the subject body (fetus) via the display screen displayed on the display unit 14. In Step S13, the calculation unit 27 sets the contours of the subject body (e.g., fetus or the like) using a set of the converted minute cubic data pieces (so-called voxels) according to the contours of the subject body (fetus) thus specified by the operator. Subsequently, the flow proceeds to Step S10. In Step S10, the voxels included within the contours of the subject body thus set, i.e., the voxels that belong to the fetus, are integrated, thereby calculating the estimated volume of the fetus. Then, the flow proceeds to Step S11 where the estimated weight of the subject body is calculated using the predetermined coefficient stored beforehand in the data storage unit 25. Thus, such an arrangement enables the estimated weight of the subject body to be appropriately calculated with high precision even if it is difficult to appropriately extract the interface between the fetus and the amniotic fluid, e.g., even if the subject body fetus in the amniotic fluid is in contact with the amnion, and there is little amniotic fluid between the fetus and the amnion.

In Step S14, the control unit 21 determines whether or not the estimated weight has been calculated. In some cases, the size of the fetus is too large, and accordingly, the three-dimensional volume data thus converted by the image reconstruction unit 26 based upon the multiple two-dimensional B-mode image data pieces and Doppler mode image data pieces cannot cover the overall region of the subject body. In this case, the estimated weight of the fetus cannot be calculated with sufficient precision. Accordingly, with such an arrangement, determination is made whether or not the estimated weight of the fetus has been calculated. In a case in which determination has been made that the estimated weight of the fetus has not been calculated, error handling processing is performed. Subsequently, the estimated weight is calculated in increments of parts (e.g., the head, the torso, etc.) of the subject body (fetus) based upon respective three-dimensional images for calculating the estimated weight.

In a case in which determination has been made in Step S14 that the estimated weight has been calculated, the flow proceeds to Step S15 where the data storage unit 25 acquires the estimated weight supplied from the calculation unit 27, and stores the estimated weight data thus acquired.

In Step S16, the data storage unit 25 supplies the estimated weight data thus stored to the DSC 28 under the control of the control unit 21. Under the control of the control unit 21, the DSC 28 acquires the estimated weight data supplied from the data storage unit 25, converts the data format of the estimated weight data thus acquired into the video scanning line format, performs predetermined image processing or computation processing for the estimated weight data thus converted, and supplies the estimated weight data thus processed to the display unit 14. The display unit 14 acquires, from the DSC 28, the data of the estimated weight of the fetus in the video scanning line format thus converted, and displays the estimated weight of the fetus on the unshown LCD or the unshown CRT based upon the data of the estimated weight of the fetus thus acquired as shown in FIG. 9.

Thus, such an arrangement allows the operator to calculate the estimated weight of the fetus at a high speed in a simple manner without troublesome operations in which the operator measures the length of each part of the fetus via the tomographic images thus displayed. Furthermore, such an arrangement prevents a two-dimensional deviation in the measurement from occurring due to an unsuitable tomographic image, e.g., a tomographic image obtained by scanning the subject body at a somewhat oblique angle with respect to the tomographic image in the axial plane. This ensures high-precision calculation of the estimated weight of the fetus. Furthermore, with such an arrangement, there is no need to repeatedly perform operations for multiple items. Accordingly, such an arrangement eliminates a situation in which the operator neglects to perform a necessary operation for a certain item from among these necessary items, thereby eliminating a situation in which the operator must perform the same operations again. Such an arrangement improves the operability of the ultrasonic diagnostic apparatus for calculating the estimated weight of the fetus.

In Step S17, the control unit 21 determines whether or not the operator has performed an operation via the input unit 13 so as to indicate a different three-dimensional image for calculating the estimated weight. That is to say, determination is made whether or not a different three-dimensional image has been indicated on the display unit 14 for calculating the estimated weight according to the operator's operation.

In a case in which determination has been made in Step S17 that a different three-dimensional image has been indicated for calculating the estimated weight in accordance with the operator's operation performed via the input unit 13, the flow returns to Step S6, and the processing following Step 6 is repeatedly performed.

Thus, such an arrangement allows the operator to repeatedly calculate the estimated weight of a fetus based upon multiple different three-dimensional images, thereby calculating the estimated weight of the fetus with high precision. Such an arrangement allows the operator to check the estimated weight of the fetus in increments of measurements performed multiple times. Thus, such an arrangement improves the operability of the ultrasonic diagnostic apparatus for calculating the estimated weight of a fetus.

In a case in which determination has been made in Step S17 that the operator has not operated the input unit 13 so as to indicate a different three-dimensional image for calculating the estimated weight of the fetus, the estimated weight calculation processing ends.

On the other hand, in a case in which determination has been made in Step S14 that the estimated weight has not been calculated, the flow proceeds to Step S18 where error handling processing is performed. Subsequently, the flow proceeds to Step S17, and the processing following Step S17 is repeatedly performed. Description has been made regarding an arrangement which allows the operator to calculate the estimated weight of a fetus based upon a single three-dimensional image. Also, an arrangement may be made in which the estimated weight is calculated in increments of parts (e.g., head, torso, etc.) of the fetus based upon multiple three-dimensional images, and the sum of the calculation results is calculated, thereby obtaining the estimated weight of the fetus. Such an arrangement allows the operator to calculate the estimated weight of the fetus at a high speed in a simple manner even if the flow proceeds to the error handling processing due to the largeness of the fetus. Thus, such an arrangement improves the operability of the ultrasonic diagnostic apparatus for calculating the estimated weight of a fetus. It should be noted that an arrangement may be made which allows the operator to set and modify the coefficients in increments of parts of the subject body (e.g., head, torso, etc.) for calculating the estimated weight. Thus, such an arrangement calculates the estimated weight of a fetus with high precision.

In a case in which determination has been made in Step S6 that the operator has not operated the estimated weight calculation button (not shown) provided to the input unit 13 so as to issue an instruction to calculate the estimated weight, the estimated weight calculation processing ends.

Description has been made regarding an arrangement in the ultrasonic diagnostic apparatus 1 according to the embodiment of the present invention, in which the estimated weight of a fetus is calculated based upon a single three-dimensional image. Also, an arrangement may be made in which the estimated weight is calculated multiple times based upon multiple three-dimensional images that differ from one another (multiple calculation results are obtained), and these multiple estimated weight calculation results are averaged, thereby calculating the estimated weight of the fetus. With such an arrangement, the estimated weight of a fetus can be calculated with higher precision.

Description has been made regarding an arrangement in the ultrasonic diagnostic apparatus 1 according to the embodiment of the present invention, in which the estimated weight of a fetus is calculated based upon a stationary three-dimensional image (frozen image). However, the present invention is not restricted to such an arrangement. For example, an arrangement may be made in which the estimated weight of a fetus is calculated based upon a real-time three-dimensional image.

A processing series described in the embodiment of the present invention may be executed by software components or hardware components.

Description has been made in the embodiment of the present invention regarding an arrangement in which the steps shown in the flowchart are executed according to the above-described procedure in a time series manner. However, the present invention is not restricted to such an arrangement. Also, these steps shown in the flowchart may be executed in parallel or executed separately, which is also encompassed by the present invention.

What is claimed is:

1. An ultrasonic diagnostic apparatus comprising:
a volume data generation unit configured to oscillate a plurality of ultrasonic wave transducer elements to transmit ultrasonic waves and to receive reflection waves which are reflected from a subject body and generate volume data on the basis of reception signals obtained by converting the reflection waves by the ultrasonic wave transducer elements;
a three-dimensional image data generation unit configured to generate three-dimensional image data on the basis of the volume data; and
an estimated weight calculation unit configured to calculate the estimated weight of the subject body on the basis of the volume data.

2. An ultrasonic diagnostic apparatus according to claim 1, further comprising a display unit configured to display the estimated weight of the subject body calculated by the estimated weight calculation unit.

3. An ultrasonic diagnostic apparatus according to claim 1, further comprising:
a contour extraction unit configured to extract the contours of the subject body on the basis of the volume data; and
an estimated volume calculation unit configured to calculate the estimated volume of the subject body on the basis of the contours of the subject body extracted by the contour extraction unit,
wherein the estimated weight calculation unit is configured to multiply the estimated volume of the subject body calculated by the estimated volume calculation unit by a predetermined coefficient set in advance, calculating the estimated weight of the subject body.

4. An ultrasonic diagnostic apparatus according to claim 3, wherein the predetermined coefficients are configured to be set in advance in respective parts of the subject body, which are used by the estimated weight calculation unit in a case of calculating the estimated weight of the subject body.

5. An ultrasonic diagnostic apparatus according to claim 3, further comprising:
a contour indicating reception unit configured to receive indicating of the contours of the subject body, in case where the contours of the subject body are extracted by the contour extraction unit; and
a contour setting unit configured to set the contours of the subject body in accordance with the indicating of the contours of the subject body received by the contour indicating reception unit,
wherein the estimated weight calculation unit is configured to calculate the estimated volume of the subject body with use of the contours of the subject body set by the contour setting unit.

6. An ultrasonic diagnostic apparatus according to claim 3, wherein the contour extraction unit is configured to determine whether or not the difference between brightness values included in the volume data is larger than a predetermined reference value set in advance, and extract the contours of the subject body on the basis of the determination result.

7. An ultrasonic diagnostic apparatus according to claim 1, wherein the estimated weight calculation unit is configured to calculate the estimated weight a plurality of times on the basis of the plural volume data generated by the volume data generation unit in the difference time phase.

8. An ultrasonic diagnostic method comprising:
a volume data generation step for oscillating a plurality of ultrasonic wave transducer elements to transmit ultrasonic waves and receiving reflection waves which are reflected from a subject body and generating volume data on the basis of reception signals obtained by converting the reflection waves by the ultrasonic wave transducer elements;
a three-dimensional image data generation step for generating three-dimensional image data on the basis of the volume data; and
an estimated weight calculation step for calculating the estimated weight of the subject body on the basis of the volume data.

9. An image processing program for an ultrasonic diagnostic apparatus, which instructs a computer to execute:
a volume data generation step for oscillating a plurality of ultrasonic wave transducer elements to transmit ultrasonic waves and receiving reflection waves which are reflected from a subject body and generating volume data on the basis of reception signals obtained by converting the reflection waves by the ultrasonic wave transducer elements;
a three-dimensional image data generation step for generating three-dimensional image data on the basis of the volume data; and
an estimated weight calculation step for calculating the estimated weight of the subject body on the basis of the volume data.
